(19) United States
(12) Patent Application Publication
(10) Pub. No.: US 2006/0276761 A1
(43) Pub. Date: Dec. 7, 2006

(54) Storage and delivery vessel for two-part medicament

(75) Inventors: Camus Kar Man Choy, Hong Kong SAR (HK); Hie Hua Wong, Hong Kong SAR (HK); Iris Frances Forster Benzie, Hong Kong SAR (HK)

Correspondence Address: LEYDIG VOIT & MAYER, LTD, 700 THIRTEENTH ST. NW, SUITE 300, WASHINGTON, DC, 20005-3960, US

(73) Assignee: The Hong Kong Polytechnic University, Kowloon (CN)

(21) Appl. No.: 11/142,396

(22) Filed: Jun. 2, 2005

(51) Int. Cl. A61M 35/00

(52) U.S. Cl. 604/295; 424/78.04

(57) ABSTRACT

A storage and delivery vessel for a two-part medicament that has first deformable chamber for holding a first part of a medicament and a second chamber for holding a second part of the medicament. The chambers are separated by a closure which opens when the first chamber is deformed by an external force, to mix the first and second parts of the medicament for dispensing through a dispensing aperture.

### BACKGROUND OF THE INVENTION

1. Field of the Invention

The invention relates to storage and delivery vessels for two part medicaments, and in particular a storage and delivery device for artificial tears.

2. Description of Prior Art

Tear fluid protects the exposed surface of the eye. In many people, especially the elderly and those who use contact lenses, tear production is low and so the use of artificial tears is common and widespread. Artificial tears are also used after ocular surgery so there is a large market for artificial tears worldwide.

Normal human tears contain antioxidants including ascorbic acid (a reduced form of vitamin C) at a concentration of around 30 μmol/l. However, ascorbic acid in solution degrades very rapidly unless the solution is highly acidic. It is undesirable to have strongly acidic artificial tears and so there is a problem with stability and viability for storage of artificial tear solutions having an antioxidant content of ascorbic acid similar to that of natural human tears.

In a wider sense, there are a number of unstable ingredients that degrade rapidly when in solution or two-part medicaments that degrade rapidly when combined.

### SUMMARY OF THE INVENTION

It is an object of the present invention to provide a storage and delivery vessel for a two-part medicament which overcomes or ameliorates the above mentioned stability problem or at least provides the public with the useful alternative.

According to a first aspect of the invention there is provided a storage and delivery vessel for a two-part medicament comprising: a first deformable chamber for holding a first part of a medicament, a second chamber for holding a second part of the medicament, a dispensing aperture, and a closure separating the first and second chambers and adapted to open or rupture when the first chamber is deformed by an external force so as to mix the first and second parts of the medicament for dispensing from the dispensing aperture.

According to a second aspect of the invention there is provided a storage and delivery device for a two-part medicament comprising a first deformable vessel for holding a first part of a medicament, a second deformable vessel for holding a second part of the medicament, a throat joining the vessels and providing a fluid passageway between the vessels, a closure located in the passageway for separating the first and second vessels and adapted to open when one of the vessels is deformed by an external force so as to mix the first and second parts of the medicament, and the vessels being separable at the throat for dispensing the medicament from the vessels.

Preferably, the closure is a rupturable impermeable membrane.

Preferably, the closure is a valve.

Preferably, the first deformable chamber holds a liquid part of the medicament and the second chamber holds a dry part of the medicament.

Preferably, the dry part of the medicament is located on an inner surface of the second chamber.

Preferably, the second chamber includes an impermeable membrane impregnated with the dry part of the medicament.

Preferably, the second chamber is deformable by an external force.

Preferably, the dispensing aperture is in the second chamber.

Further aspects of the invention will become apparent from the following drawings and description.

### BRIEF DESCRIPTION OF THE DRAWINGS

Embodiments of the invention will now be described by way of example only and with reference to the accompanying drawings in which:

FIG. 1 is a first embodiment of a storage and delivery vessel for a two-part medicament according to the invention,

FIG. 2 shows the chambers of the storage and delivery vessel separated,

FIGS. 3 to 6 illustrate use of the storage and delivery vessel,

FIG. 7 show a valve closure for separating the chambers of the vessel, and

FIG. 8 is a second embodiment of a storage and delivery vessel for a two-part medicament according to the invention, with vessels separated by a throat.

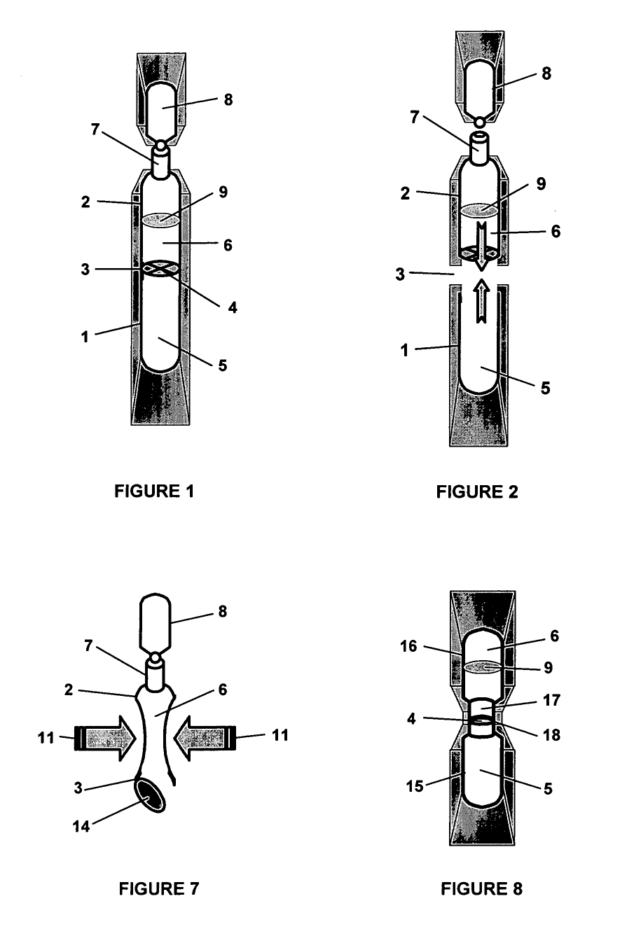

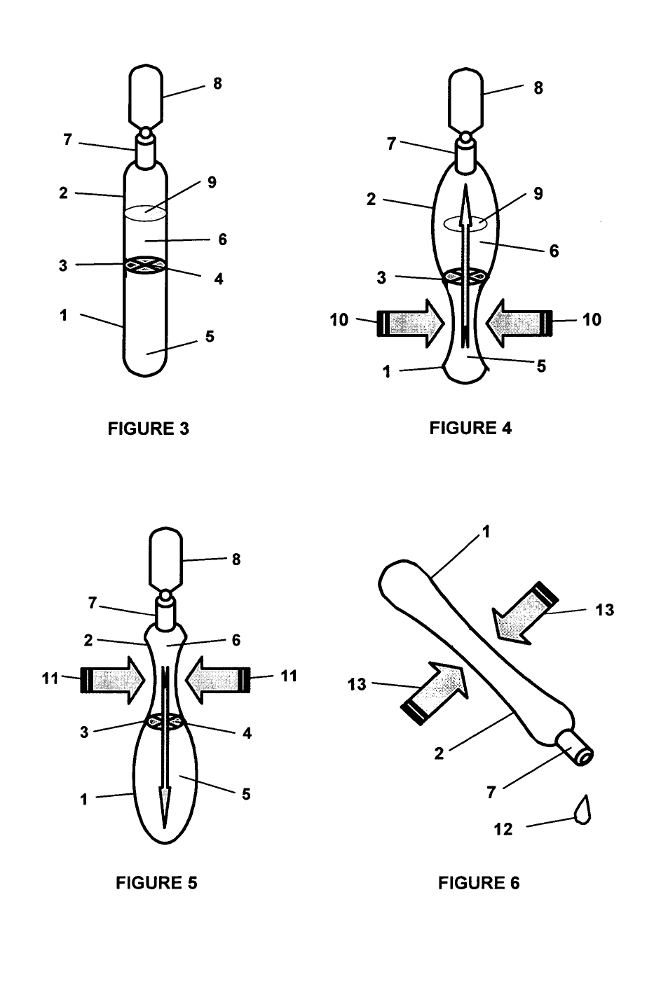

### DESCRIPTION OF THE PREFERRED EMBODIMENTS

In drawings 1 to 6 there is depicted a storage and delivery vessel for a single application of artificial tears. The vessel comprises a first deformable vessel half 1 connected to a second deformable vessel half 2 at a waist section 3. The vessel halves 1, 2 are connected by either a threaded joint or a friction fit male/female coupling arrangement. A closure in the form of a rupturable impermeable membrane 4 is provided across the waist section 3 defining a first chamber 5 within the first vessel half 1 and a second chamber 6 within the second vessel half 2. At one end of the second vessel half 2 is a dispensing spout 7 with a closure plug 8.

The first chamber 5 contains a physiologically balanced salt solution. The second chamber 6 contains a permeable membrane 9 impregnated with ascorbic acid in dry form. The permeable membrane 9 is impregnated by placing on it a droplet of high concentration ascorbic acid in liquid form in a low humidity environment. The low humidity causes the droplet to dry almost immediately without significant degradation of the ascorbic acid. The membrane 9 is then transferred to the second chamber 6 of the device.

To use the artificial tears the user squeezes the first vessel half 1 between their fingers as illustrated by arrows 10 in FIG. 4. The rupturable membrane 4 is ruptured and the physiologically balanced salt solution is forced through the waist 3 into the second chamber 6. The physiologically balanced salt solution wets the impermeable membrane 9 becoming infused with ascorbic acid. To fully mix the physiologically balanced salt solution and ascorbic acid the user can repeatedly deform the second vessel half 2 between their fingers in the direction of arrows 11 and first vessel half 1 in the direction of arrows 10 to transfer the artificial tears backwards and forwards between the two chambers 5 and 6.

The freshly mixed artificial tear 12 is dispensed into the eye by removing the closure 8 from spout 7 and squeezing the vessel between fingers in the direction of arrows 13.

The present invention allows for the storage and delivery of a single dose of artificial tears in a composition that more closely resembles that of natural human tears but which does not degrade with storage. However, the skilled-addressee will recognise that the storage and delivery vessel can be used for other two-part medicaments that are preferably kept separated and mixed immediately prior to use.

Referring to FIG. 7, in other embodiments of the invention the rupturable membrane 4 is replaced with a flap valve 14 in the waist 3. The flap valve 14 is fixedly attached to the waist 3 at a single point and is forced open by the solution when first vessel half 1 is squeezed.

Referring to FIG. 8, a second embodiment of the invention comprising two vessels 15 and 16 connected to a throat 17. The throat 17 provides a fluid passageway between the two vessels 15, 16. The first vessel defines the first chamber 5 and the second vessel defines the second chamber 6. The rupturable membrane 4 is provided in the passageway for separating the two vessels 15, 16. The rupturable membrane 4 is ruptured as described above and the contents of the two chambers 5, 6 mixed accordingly. Throat 17 is provided with a scored collar 18 allowing a user to twist the vessels 15, 16 in opposite directions to separate the vessels at the throat 17. The opened passageway provides a dispensing aperture for dispensing the artificial tear into an eye.

Where in the foregoing description reference has been made to integers or elements having known equivalents then such are included as if individually set forth herein.

Embodiments of the invention have been described, however it is understood that variations, improvement or modifications can take place without departure from the spirit of the invention or scope of the appended claims.

1. A storage and delivery vessel for a two-part medicament comprising:
a first deformable chamber for holding a first part of a medicament,
a second chamber for holding a second part of the medicament,
a dispensing aperture, and
a closure separating the first and second chambers and opening or rupturing when the first chamber is deformed by an external force, as to mix the first and second parts of the medicament for dispensing from the dispensing aperture.

2. The vessel of claim 1 wherein the closure is a rupturable impermeable membrane.

3. The vessel of claim 1 wherein the closure is a valve.

4. The vessel of claim 1 wherein the first deformable chamber holds a liquid part of the medicament and the second chamber holds a dry part of the medicament.

5. The vessel of claim 4 wherein the dry part of the medicament is located on an inner surface of the second chamber.

6. The vessel of claim 4 wherein the second chamber includes an impermeable membrane impregnated with the dry part of the medicament.

7. The vessel of claim 1 wherein the second chamber is deformable by an external force.

8. The vessel of claim 1 wherein the dispensing aperture is in the second chamber.

9. A storage and delivery device for a two-part medicament comprising:
a first deformable vessel for holding a first part of a medicament,
a second deformable vessel for holding a second part of the medicament,
a throat joining the first and second vessels and providing a fluid passageway between the first and second vessels, and
a closure located in the passageway for separating the first and second vessels and opening when one of the first and second vessels is deformed by an external force, to mix the first and second parts of the medicament, the first and second vessels being separable at the throat for dispensing the medicament from the first and second vessels.

10. The device of claim 9 wherein the closure is a rupturable impermeable membrane.

11. The device of claim 9 wherein the closure is a valve.

12. The device of claim 9 wherein the first vessel holds a liquid part of the medicament and the second vessel holds a dry part of the medicament.

13. The device of claim 12 wherein the dry part of the medicament is located on an inner surface of the second vessel.

14. The device of claim 12 wherein the second vessel includes an impermeable membrane impregnated with the dry part of the medicament.
